SEQUENCE LISTING (3 sequences)

SEQ ID NO: 1 - DNA, 21 bp, Artificial Sequence
  oligonucleotide
accttaagagcccacggttcc

SEQ ID NO: 2 - DNA, 13 bp, Artificial Sequence
  oligonucleotide
aaagaatataccc

SEQ ID NO: 3 - DNA, 46 bp, Artificial Sequence
  oligonucleotide
  misc_feature (22)..(33): n is a, c, g, or t
accttaagagcccacggttccnnnnnnnnnnnnaaagaatataccc

(19) United States
(12) Patent Application Publication
(10) Pub. No.: US 2022/0034881 A1
(43) Pub. Date: Feb. 3, 2022

(54) BARCODED PEPTIDE-MHC COMPLEXES AND USES THEREOF

(71) Applicant: BRISTOL-MYERS SQUIBB COMPANY, Princeton, NJ (US)

(72) Inventors: Kalpit Shah, San Mateo, CA (US); Kandasamy Ravi, Bryn Mawr, PA (US)

(21) Appl. No.: 17/278,966

(22) PCT Filed: Sep. 23, 2019
(86) PCT No.: PCT/US2019/052376

§ 371 Date: Mar. 23, 2021

Related U.S. Application Data
(60) Provisional application No. 62/735,803, filed on Sep. 24, 2018.

(51) Int. Cl. G01N 33/569; C12Q 1/6804; C12N 15/10

(52) U.S. Cl. CPC G01N 33/56977; C12N 15/1065; C12Q 1/6804

(57) ABSTRACT

In certain embodiments, the present disclosure provides methods combining (i) screening with DNA-barcoded peptide-major histocompatibility complex (pMHC) to detect T lymphocytes specific for these peptides, and (ii) single-cell sequencing of the T lymphocytes identified in the screening to analyze their transcriptome.

### CROSS-REFERENCE TO RELATED APPLICATIONS

This application claims the priority benefit of U.S. Provisional Application No. 62/735,803, filed Sep. 24, 2018, which is incorporated herein by reference in its entirety.

### FIELD OF THE INVENTION

The present invention relates to T-cell epitome mapping and transcriptome analysis.

### BACKGROUND

T cells play a vital role in countering viral infections and tumors. T cells get activated via interaction between T-cell receptors (TCRs) and peptide-major histocompatibility complex (pMHC). The interaction between TCRs and pMHCs may induce proliferation, development of effector phenotype including cytokine release. Hence, identification of the peptides (antigens) recognized by individual T cells and characterizing peptide-specific T cells is essential for understanding and treating immune-related diseases.

Currently, mass cytometry-based (Newell et al. (2013) Nature Biotechnology, 623-629), fluorescence-based (Altman (1996) Science, 94-96) or double-stranded DNA barcode-based approaches (Bentzen et al. (2016) Nature Biotechnology, 1037-1045) are limited to the identification of the antigens recognized by individual T cells with a limited flow-based characterization of the antigen-specific T cells. Similarly, DNA barcode antibody labeling strategies (Stoeckius et al. (2017) Nature Methods, 865-868; Peterson et al. (2017) Nature Biotechnology, 936-939) is limited to simultaneous measurement of gene expression and cell surface epitope expression.

### SUMMARY OF THE INVENTION

The present disclosure provides a method of simultaneous T-cell epitope mapping and/or transcriptome characterization at single cell resolution in a sample comprising T-cells, the method comprising: (a) labeling each unique peptide-major histocompatibility complex (pMHC) with a unique barcode, thereby yielding a population of barcoded pMHC constructs; (b) contacting the sample comprising T-cells with the population of barcoded pMHC constructs, wherein at least one T cell receptor on a T-cell binds to at least one of the barcoded pMHC constructs (“T cell receptor epitope”); and, (c) sequencing the T-cells using single cell sequencing, wherein the single cell sequencing simultaneously identifies the T-cell receptor epitopes and transcriptome genes in each T-cell.

The present disclosure also provides a method of simultaneous T-cell epitope mapping and/or transcriptome characterization in a single T-cell obtained from a sample, the method comprising (a) labeling each unique peptide-major histocompatibility complex (pMHC) with a unique barcode, thereby yielding a population of barcoded pMHC constructs; (b) contacting a T-cell with the population of barcoded pMHC constructs, wherein a T cell receptor on the T-cell binds to at least one of the barcoded pMHC constructs (“T cell receptor epitope”); and, (c) sequencing the T-cell using a single cell sequencing, wherein the single cell sequencing simultaneously identifies the T-cell receptor epitope and transcriptome genes in the T-cell.

In some aspects, the single cell sequencing is a droplet-based single cell sequencing. In some aspects, each droplet of the sequencing comprises (i) the T-cell labelled by at least one barcoded pMHC construct in (b); and (ii) a primer bead comprising primers for transcriptome measurement.

In some aspects, each barcode is a single stranded nucleic acid. In some aspects, the single stranded nucleic acid is DNA. In some aspects, each barcode comprises a unique sample identification sequence. In some aspects, the sample identification sequence is designed based on Hamming codes. In some aspects, the sample identification region is at least 10 bp, at least 11 bp, at least 12 bp, at least 13 bp, at least 14 bp, at least 15 bp, at least 16 bp, at least 17 bp, at least 18 bp, at least 19 bp, at least 20 bp, at least 21 bp, at least 22 bp, at least 23 bp, at least 24 bp, at least 25 bp, at least 26 bp, at least 27 bp, at least 28 bp, at least 29 bp or at least 30 bp long. In some aspects, the sample identification region is between 10 bp and 30 bp, between 11 bp and 29 bp, between 12 bp and 28 bp, between 13 [symbol] 14 bp and 26 bp, between 15 bp and 25 bp, between 16 bp and 24 bp, between 17 bp and 23 bp, or between 18 bp and 22 bp.

In some aspects, the sample identification region is flanked by two constant regions (a 5′ constant region and a 3′ constant region). In some aspects, the 5′ constant region is used for PCR amplification and for annealing to an index primer. In some aspects, the index primer comprises a unique molecular index (UMI). In some aspects, the UMI comprises an Illumina i7 UMI. In some aspects, the 3′ constant region anneals to a template-switch oligo in a droplet based single cell sequencing platform.

In some aspects, the template-switch oligo comprises a 10X cell barcode or a Dropseq cell barcode. In some aspects, each barcoded pMHC construct comprises a scaffold. In some aspects, the scaffold comprises neutravidin. In some aspects, the scaffold comprises a dextran. In some aspects, each barcoded pMHC construct comprises 4 identical pMHC monomers attached to a neutravidin scaffold. In some aspects, each barcoded pMHC construct comprises 5 identical pMHC monomers attached to a dextran scaffold.

In some aspects, the sample comprising T lymphocytes is peripheral blood, cord blood, tissue biopsies, or liquid biopsies. In some aspects, the 5′ constant region comprises the nucleic acid sequence as set forth in SEQ ID NO: 1 (ACCTTAAGAGCCCACGGTTCC). In some aspects, the 3′ constant region comprises the nucleic acid sequence as set forth in SEQ ID NO: 2 (AAAGAATATACCC).

The present disclosure also provides a T-cell epitope identified by any one of the methods disclosed herein. Also provided is a transcriptome of a T cell identified by any of the methods disclosed herein.

The present disclosure also provides a DNA barcoded pMHC construct comprising at least one pMHC peptide covalently or non-covalently attached to a scaffold molecule, and at least one barcode covalently or non-covalently attached to the scaffold. In some aspects, the scaffold molecule is neutravidin or dextran. In some aspects, the DNA barcode comprises SEQ ID NO: 3.

Also provided is a method of method of manufacturing a DNA barcoded pMHC construct of the present disclosure comprising (1) attaching a 4 or 5 pMHC peptides to a scaffold, wherein the scaffold is dextran or neutravidin; and (2) attaching at least one DNA barcode to the scaffold, wherein the DNA barcode comprises SEQ ID NO: 3.

### BRIEF DESCRIPTION OF THE DRAWINGS

FIG. 1 shows two alternative DNA barcode labeling strategies. These DNA-barcoded constructs comprising pMHC can be used to identify antigen-specific T cells during single cell sequencing protocol. In one strategy, the DNA-barcoded pMHC construct is a pMHC tetrameric construct comprising a neutravidin scaffold. In the alternative strategy, the DNA-barcoded pMHC construct is a pMHC multimeric construct (e.g., pentameric) comprising a dextran scaffold.

FIG. 2 shows the strategy for generating a DNA-barcoded library of pMHC multimers such that each DNA barcode corresponds to a unique pMHC multimer.

FIG. 3 is a schematic representation showing a droplet for sequencing comprising a T-cell, a DNA-barcoded pMHC construct (in particular, a neutravidin complex), and a sequencing primer bead for transcriptome analysis that would contain a series of primers corresponding to each one of the genes to be amplified as part of the transcriptome analysis.

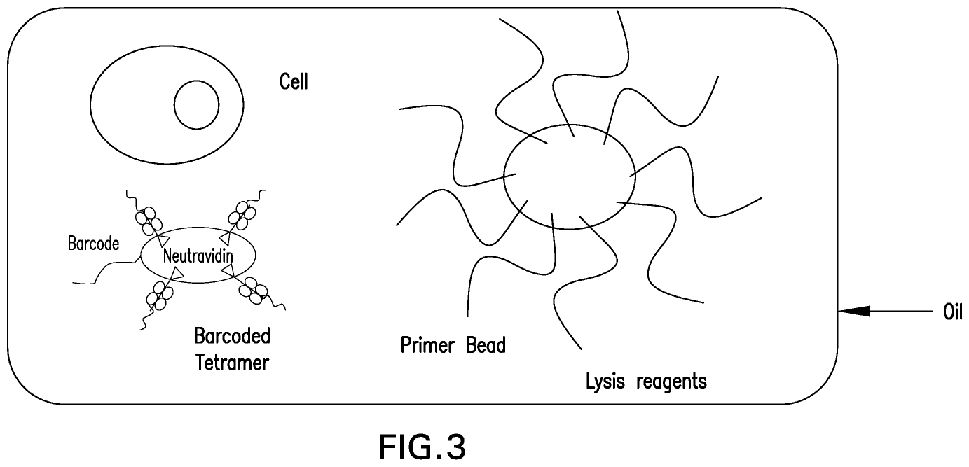

### DETAILED DESCRIPTION OF THE INVENTION

The present disclosure provides methods that allow the simultaneous detection of antigen-specific T cells and the measurement of their transcriptome at single cell level. The disclosed methods use peptide-major histocompatibility complex (pMHC) constructs comprising a scaffold comprising several pMHC monomers attached to the scaffold, as well as a DNA barcode. Each unique barcodes biunivocally relates to a unique pMHC.

These methods, by combining single cell sequencing with pMHC barcoded with specific single-stranded DNAs, allowing for the rapid and simultaneous identification and quantification of specific T-cell receptors on the cell surface and for the transcriptomic characterization of T lymphocytes. These methods can be used, for example, for screening peptide specificity, simultaneous characterization of the transcriptome of diverse antigen-specific T cells at a single cell level, validating the results from the T-Cell receptor (TCR) sequencing, diagnostic tests, predicting the efficacy of immune therapies, or measuring the immune reactivity after vaccination or immune therapy.

Furthermore, the methods disclosed herein can also be used to identify and characterize rare cell types based on their affinity to ligands.

Accordingly, the present disclosure provides a method of simultaneous T-cell epitope mapping and/or transcriptome characterization at single cell resolution in a sample [symbol] the method comprising (a) labeling each unique peptide-major histocompatibility complex (pMHC) with a unique barcode, thereby yielding a population of barcoded pMHC constructs; (b) contacting the sample comprising T-cells with the population of barcoded pMHC constructs, wherein at least one T cell receptor on a T-cell binds to at least one of the barcoded pMHC constructs (“T cell receptor epitope”); and, (c) sequencing the T-cells using single cell sequencing, wherein the single cell sequencing simultaneously identifies the T-cell receptor epitopes and transcriptome genes in each T-cell.

As used herein, the term “unique” refers to a biunivocal relationship between the DNA barcode and the pMHC conjugated to the barcode or to the construct comprising the barcode. Thus, the term unique means that a specific DNA barcode corresponds to a specific pMHC and only that specific pMHC, and that specific pMHC corresponds to a specific DNA barcode and only that specific DNA barcode.

As used herein, the terms “at single cell resolution” or “at single cell level” means that the samples comprises a population of T cells, but each set of T epitope mapping data and transcriptome data obtained in each single cell sequencing reaction corresponds to a single cell.

In some aspects, the single cell sequencing is a droplet-based single cell sequencing. However, other sequencing methods allowing the sequencing of a single cell in another compartment (e.g., a bead, an array well, etc.) can also be used to practice the methods disclosed herein. The methods disclosed herein can also be practicing by using sequencing methods that, although do not sequence a single cell, allow the multiplexed sequencing of a number of cells wherein each cell is uniquely identified (e.g., by barcoding).

In some aspects, each droplet of the sequencing comprises (i) the T-cell labelled by at least one barcoded pMHC construct in (b); and (ii) a primer bead comprising primers for transcriptome measurement. As discussed above, the sequencing reaction may be conducted using an alternative system is which the reactants are confined, e.g., in an array well, a capillar or compartment in a microfluidic systems, a bead, a liposome, etc. Furthermore, the primers for transcriptome measurement can be bound to an alternative scaffold or container, for example, a dendrimer, a linear or branched polymer, a well, a droplet, etc. [symbol] cts, each barcode is a single stranded nucleic acid. In other aspects of the present disclosure, the nucleic acid can be, e.g., double stranded or branched. In some aspects, the nucleic acid, e.g., a single stranded nucleic acid, is a DNA or an RNA. In some aspects, the nucleic acid can comprise, e.g., non-natural nucleobases (e.g., LNA) and/or non-natural backbone linkages (e.g., phosphorothioate). In some aspects, the barcode can comprise an universal base. In some aspects, each barcode comprises a unique sample identification sequence.

In some aspects, the sample identification sequence is designed based on Hamming codes. In some aspects, alternative codes can be used in the sample identification sequence. In some aspects, the code is an error-correcting code, e.g., a code comprising a hash function. In some aspects, the hash function is, e.g., a repetition code, a parity bit, or a checksum.

In some aspects, the sample identification region is at least 10 bp, at least 11 bp, at least 12 bp, at least 13 bp, at least 14 bp, at least 15 bp, at least 16 bp, at least 17 bp, at least 18 bp, at least 19 bp, at least 20 bp, at least 21 bp, at least 22 bp, at least 23 bp, at least 24 bp, at least 25 bp, at least 26 bp, at least 27 bp, at least 28 bp, at least 29 bp or at least 30 bp long. In some aspects, the sample identification region is between 10 bp and 30 bp, between 11 bp and 29 bp, between 12 bp and 28 bp, between 13 and 27 bp, between 14 bp and 26 bp, between 15 bp and 25 bp, between 16 bp and 24 bp, between 17 bp and 23 bp or between 18 bp and 22 bp.

In some aspects, the sample identification region is flanked by two constant regions (a 5′ constant region and a 3′ constant region). In some aspects, the 5′ constant region is used for PCR amplification and for annealing to an index primer. In some aspects, the 3′ constant region anneals to a template-switch oligo in a single cell sequencing platform, e.g., a droplet based single cell sequencing platform. In some aspects, the flanking constant regions are transposed, i.e., the 3′ constant region is used for PCR amplification and for annealing to an index primer, and the 5′ constant region anneals to a template-switch oligo in a single cell sequencing platform, e.g., a droplet based single cell sequencing platform.

In some aspect, the index primer comprises a unique molecular index (UMI). Unique molecular identifiers (UMIs) are short sequences or “barcodes” added to each read in some next generation sequencing protocols. They serve to reduce the quantitative [symbol] DNA amplification, which is necessary to get enough reads for detection. In some specific aspects, the UMI comprises an Illumina i7 UMI.

In some aspects, template-switch oligo comprises a 10X cell barcode or a Drop-Seq cell barcode. Template-switching polymerase chain reaction (TS-PCR) is a method of reverse transcription and polymerase chain reaction (PCR) amplification that relies on a natural PCR primer sequence at the polyadenylation site and adds a second primer through the activity of murine leukemia virus reverse transcriptase. For example, in Drop-Seq, by using syringe pumps to transmit a steady rate of isolated cells and uniquely barcoded beads, it is possible to isolate individual cells and beads together in droplets of lysis buffer, where the polyadenylation site binds to a bead-specific primer containing a unique identifying sequence. This primer also contains a common sequence upstream of the identifier, so that after it is extended by reverse transcription, subsequent rounds of PCR will incorporate the tag, which permits each isolated cDNA that is sequenced to be tracked back to a specific originating bead. This permits the relative levels of transcripts in many individual cells to be analyzed simultaneously, creating, e.g., a rational basis for the classification of these cells into particular cell types.

In some aspects, each barcoded pMHC construct comprises a scaffold. A person of skill in the art would understand that in addition to the scaffolds disclosed herein there are numerous scaffold molecules known in the art that may be used to practice the claim invention (e.g., instead of dextran, other polymers may be used).

In some specific aspects, the scaffold comprises neutravidin. Neutravidin protein is a deglycosylated version of avidin, with a mass of approximately 60,000 daltons. As a result of carbohydrate removal, lectin binding is reduced to undetectable levels, yet biotin binding affinity is retained because the carbohydrate is not necessary for this activity. Avidin has a high pI but Neutravidin has a near-neutral pI (pH 6.3), minimizing non-specific interactions with the negatively-charged cell surface or with DNA/RNA. Neutravidin still has lysine residues that remain available for derivatization or conjugation. Like avidin itself, Neutravidin is a tetramer with a strong affinity for biotin (Kd=10-15 M).

In some specific aspects, the scaffold comprises a dextran. Dextran is a complex branched glucan (polysaccharide derived from the condensation of glucose. IUPAC defines dextran as “Branched poly-α-d-glucosides of microbial origin having glycosidic bonds predominantly C-1→C-6”.[1] Dextran chains are of varying lengths (from 3 to 2000 [symbol] lymer main chain consists of α-1,6 glycosidic linkages between glucose monomers, with branches from α-1,3 linkages.

In some aspects, each barcoded pMHC construct comprises 4 identical pMHC monomers attached to a neutravidin scaffold. In other aspects, the barcoded pMHC comprises 1, 2, 3, 4 pMHC monomers attached to a neutravidin scaffold.

In some aspects, each barcoded pMHC construct comprises 1, 2, 3, 4, 5, 6, 7, 8, 9 or 10 pMHC monomers attached to a dextran scaffold. In some aspects, each barcoded pMHC construct comprises 5 identical pMHC monomers attached to a dextran scaffold.

In some aspects, the sample comprising T lymphocytes, e.g., a peripheral blood sample, a cord blood sample, a tissue biopsy sample, a liquid biopsy sample, or a combination thereof. In some aspects, the sample comprises purified or partially purified T lymphocytes. In some aspects, the sample is a pooled sample. In some aspects, the pooled sample comprises multiple samples from the same individual. In some aspects, the pooled sample comprises multiple samples from multiple individuals. In some aspects, all the samples pooled from multiple individuals are the same type of sample.

In some aspects, the sample or samples are obtained from a human subject. In other aspects, the sample or samples are obtained from an animal. In some aspects, the sample or samples are obtained from an animal model, e.g., a mouse, a rat, or a non-human primate. In some aspect, the sample or samples are obtained from a cell line.

In some aspects, the 5′ constant region sequence used for PCR amplification and for annealing to an index primer comprises the nucleic acid sequence as set forth in SEQ ID NO: 1 (ACCTTAAGAGCCCACGGTTCC). In some aspects, the 3′ constant region sequence that anneals to a template-switch oligo in a single cell sequencing platform comprises the nucleic acid sequence as set forth in SEQ ID NO: 2 (AAAGAATATACCC).

In present disclosure also provides a T cell epitope identified by any of the methods disclosed herein. Also provided is a transcriptome of a T cell identified by any of the methods disclosed herein.

In some aspects the present disclosure, the identification of T cell epitopes can be qualitative. In other aspects, the identification of T cell epitopes can be quantitative. In some aspects, the genes identified in the transcriptome analysis are determined qualitatively. In some aspects, the genes identified in the transcriptome analysis are determined quantitatively. [symbol] acts, the T cell epitope data and/or transcriptome data obtained when applying the methods disclosed herein can be used as biomarkers. The presence or absence of the biomarkers, their quantities with respect to one or more thresholds, their increase or decrease with respect to one or more control, or combinations thereof can be used to, e.g.,
(i) Stratify a population of subjects,
(ii) Determine the prognosis of a subject,
(iii) Treat a subject with a certain therapeutic agent,
(iv) Discontinue the treatment of a subject with a certain therapeutic agent,
(v) Modify the treatment on a subject with a certain therapeutic agent,
(vi) Determine the response of a subject to a therapeutic agent or lack thereof,
(vii) Select a patient for treatment with a therapeutic agent,
(viii) Determine the efficacy of a therapeutic agent,
(ix) Screen therapeutic agents to determine their efficacy to treat a disease or condition, or
(x) Any combination thereof.

The present disclosure also provides barcoded peptide constructs that can be used to practice the methods disclosed above. These barcoded peptide constructs comprise (a) a scaffold (e.g., a neutravidin or a dextran scaffold), (b) a population of peptides covalently or non-covalently attached to the scaffold (e.g., pMHC), and (c) at least one nucleic acid barcode covalently or non-covalently attached to the scaffold (e.g., a DNA barcode). In some aspects, the barcoded peptide constructs disclosed herein comprise at least one pMHC and at least one DNA bar code comprising at least one sample identification sequence and at least one PCR amplification primer.

In some aspects, the barcoded peptide constructs of the present disclosure can comprise peptides other than pMHC, e.g., non-pMHC peptides binding to a surface receptor in T lymphocytes which is not a TCR. Also, in some aspects, the barcoded peptide constructs of the present disclosure can target lymphocytes other than T lymphocytes, or even cells that are not lymphocytes. Thus, in general, the barcoded peptide constructs disclosed herein can be used to generate barcoded libraries comprising peptides binding to a receptor or receptor on the surface of a certain cell, wherein binding of a specific barcoded peptide construct to a specific receptor molecule on the surface of the cell can be used to identify the presence of a certain type of a receptor or to identify or characterize the [symbol] ptor for a certain ligand or ligand variant. As disclosed above, the identification and characterization of a certain surface receptor by using a barcoded peptide construct of the present disclosure can be quantitative and/or qualitative.

The preset disclosure also provides methods to manufacture the barcoded peptide constructs disclosed herein. In some aspect. The method of manufacture comprises covalently or non-covalently attaching at least one peptide (e.g., a pMHC) to a scaffold molecule (e.g., neutravidin), and covalently or non-covalently attaching at least one barcode (e.g., a DNA barcode disclosed herein) to the scaffold.

In a specific aspect, the method of manufacture comprises covalently attaching a DNA barcode of SEQ ID NO: 3 to a neutravidin scaffold and non-covalently attaching 4 identical pMHC monomers to the neutravidin scaffold.

In another specific aspect, the method of manufacture comprises non-covalently attaching a DNA barcode of SEQ ID NO: 3 to a dextran scaffold and non-covalently attaching 5 identical pMHC monomers to the dextran scaffold.

The present invention is further illustrated by the following examples which should not be construed as further limiting. The contents of all figures and all references, patents and published patent applications cited throughout this application are expressly incorporated herein by reference.

### Example 1

### Generation of DNA Barcodes and Neutravidin or Dextran Conjugates

The methods disclosed herein use specific DNA barcode which allows for simultaneous identification of the antigen-specific T lymphocytes and the characterization of their transcriptome at a single cell level using droplet-based sequencing methodologies.

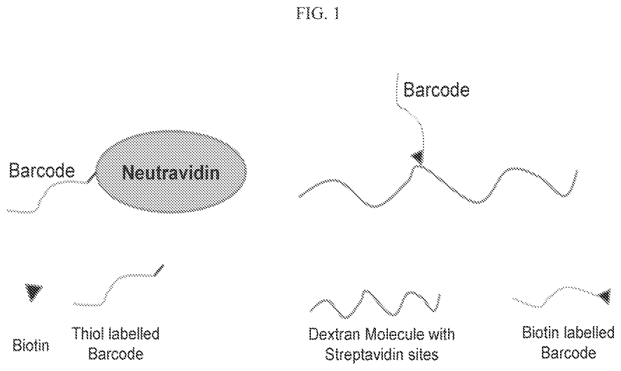

The DNA barcode can be synthesized with either a 5′ biotin tag or a 5′ Thiol modifier and attached to the surface of Streptavidin-Dextran or neutravidin respectively (FIG. 1). For conjugation with streptavidin-Dextran, the titrated amounts of 5′ modified DNA barcode allows for an estimated one DNA barcode per dextran backbone. The DNA barcode consists of the 12 bp sample identification sequences designed based on Hamming codes flanked with two constant regions: [symbol] GCCCACGGTTCC 3′ (SEQ ID NO: 1) (5′ end of the DNA barcode, used for PCR amplification and for annealing to the Illumina i7 index primer). 5′AAAGAATATA CCC 3′ (SEQ ID NO: 2) (3′ end of the DNA barcode, used for annealing to template-switch oligo in 10X single cell or other droplet based single cell platforms).

The middle 12 nucleotides (marked with “nnnnnnnnnnnn”) of the barcode designate the tetramer DNA barcode that is specific for each epitope. This sequence can be used to deconvolute the reads after the sequencing. For identification of multiple epitopes one can design specific barcode for each epitope of interest that can be pooled, therefore enabling multiplexing during incubation with single cell suspensions. Hence the complete DNA barcode will be

(SEQ ID NO: 3)
5′-biotin/5′-Thiol-ACCTTAAGAGCCCACG
GTTCCnnnnnnnnnnnnAAAGAATATACCC-3′.

### Example 2

### Construction of a Library of DNA-Barcoded pMHC Multimers

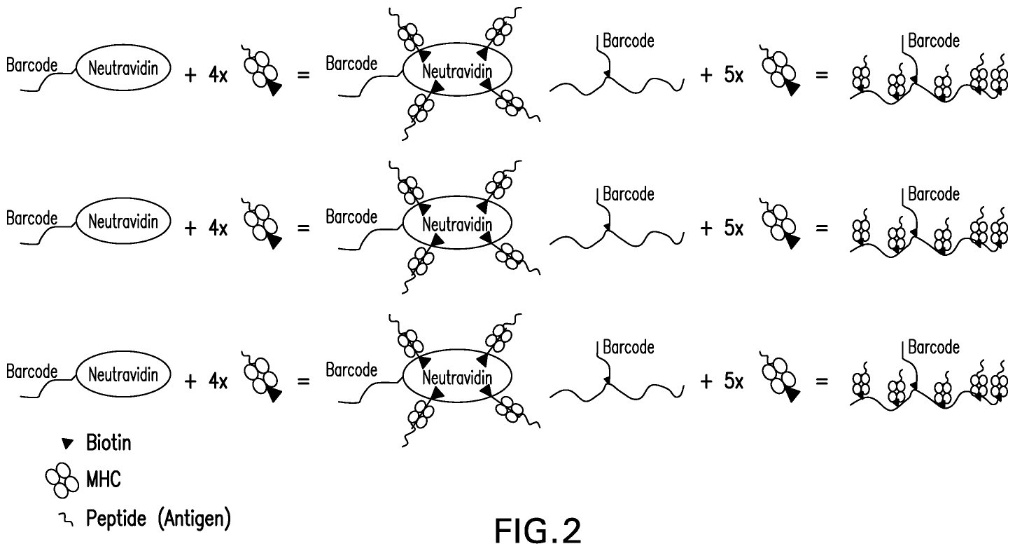

Chemically synthesized or commercially available peptides are used to synthesize biotinylated MHC/peptide complexes as described in Garboczi et al. (1992) PNAS, 3429-3433. Four or five biotinylated pMHC monomers are conjugated to the unoccupied SA-binding sites on the DNA barcoded-dextran or DNA barcoded-neutravidin. The DNA-barcoded pMHC multimers (“pMHC constructs”) are pooled such that each DNA barcode codes for a different pMHC multimers. FIG. 2 describes the process schematically.

The strategy of using DNA barcode labeled-neutravidin as the core rather than streptavidin for pMHC tetramers offers the advantage of lowest nonspecific binding amongst the known biotin-binding proteins.

The final library consists of different pMHC-multimers (“pMHC constructs”), each one of them coded by a unique DNA barcode. The DNA barcode is utilized in this approach as a beacon for identifying T lymphocytes specific for a particular pMHC multimer. Each specific tetramer read will indicate expression of specific epitope on the cell surface.

### Example 3

### Staining of Antigen-Specific T Cells with pMHC Multimer Barcoded with DNA

Staining of antigen-specific T Cells with pMHC multimer barcoded with DNA for simultaneous detection and characterization of antigen-specific T cells using droplet based sequencing technologies.

A single cell suspension from peripheral blood, cord blood, tissue biopsies, liquid biopsies, or any other cells consisting of T lymphocytes can be collected and washed twice in phosphate buffer saline. Following which cells are washed with ice-cold blocking buffer (2% BSA, 0.01% Tween-20 or another low ionic reagent, and 10% FBS). The non-specific interactions are blocked by incubating cells with commercially available Fc Receptor blocking buffer for 10 minutes on ice. Following blocking, cells are stained by incubating them with the pMHC multimer library coded with DNA barcodes for 30-minutes on ice.

Following the staining, non-specific interactions between T cell receptors and MHC multimers, as well as free-floating MHC multimers, are removed by washing cells 5 times in ice-cold blocking buffer. An appropriate number of cells are resuspended in the phosphate buffer saline for loading on 10X genomics single cell platform or other droplet-based systems. The cDNA is synthesized alongst with the addition of a unique cell barcode coding each cell encapsulated in an oil droplet.

Following cDNA synthesis, the product is amplified using 10X primers, or custom primers in case of custom droplet-based sequencing. To ensure sufficient amplification of barcodes linked with pMHC multimers, we also add custom primer (ACCTTAAGAGCCCACGGTTCC). Both gene expression cDNA library and pMHC multimer DNA barcode library is amplified and indexed for sequencing using next-generation sequencing technologies.

The sequenced reads will be demultiplexed to obtain fastq files.

The following features will be extracted from them:
1. 10X or Dropseq Cell barcode—Used to identify cells where reads came from
2. Unique Molecular Index (UMI) —For identifying reads coming from PCR amplification.
3. Sequencing Reads—Reads that come from genes.
4. DNA barcode from pMHC-multimer—To identify reads from pMHC multimer. [symbol] tion extracted from fastq files is used to construct matrices where the row corresponds to the genes or barcodes linked with pMHC multimer. The columns within these matrices correspond to the cell barcode. The strategy presented here not only allows to screen a large pool of antigens, identify receptors on the surface of T lymphocytes, identify antigen-specific T lymphocytes and to study their transcriptome at a single cell level.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer [symbol] in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A method of simultaneous T-cell epitope mapping and/or transcriptome characterization at single cell resolution in a sample comparing T-cells, the method comprising:
(a) labeling each unique peptide-major histocompatibility complex (pMHC) with a unique barcode, thereby yielding a population of barcoded pMHC constructs;
(b) contacting the sample comprising T-cells with the population of barcoded pMHC constructs, wherein at least one T cell receptor on a T-cell binds to at least one of the barcoded pMHC constructs (“T cell receptor epitope”); and
(c) sequencing the T-cells using single cell sequencing,
wherein the single cell sequencing simultaneously identifies the T-cell receptor epitopes and transcriptome genes in each T-cell.

2. The method of claim 1, wherein the single cell sequencing is a droplet-based single cell sequencing.

3. The method of claim 2, wherein each droplet of the sequencing comprises:
a) the T-cell labelled by at least one barcoded pMHC construct in (b); and
b) a primer bead comprising primers for transcriptome measurement.

4. The method of claim 1, wherein each barcode is a single stranded nucleic acid.

5. The method of claim 4, wherein the single stranded nucleic acid is DNA.

6. The method of claim 1, wherein each barcode comprises a unique sample identification sequence.

7-8. (canceled)

9. The method of claim 6, wherein the sample identification region is flanked by two constant regions (a 5′ constant region and a 3′ constant region).

10. The method of claim 9, wherein the 5′ constant region is used for PCR amplification and for annealing to an index primer.

11. The method of claim 10, wherein the index primer comprises a unique molecular index (UMI).

12. The method of claim 11, wherein the UMI comprises an Illumina i7 UMI.

13. The method of claim 9, wherein the 3′ constant region anneals to a template-switch oligo in a droplet based single cell sequencing platform.

14. The method of claim 13, wherein the template-switch oligo comprises a 10X cell barcode or a Dropseq cell barcode.

15. The method of claim 1, wherein each barcoded pMHC construct comprises a scaffold.

16. The method of claim 15, wherein the scaffold comprises neutravidin.

17. The method of claim 15, wherein the scaffold comprises a dextran.

18. The method of claim 16, wherein each barcoded pMHC construct comprises 4 identical pMHC monomers attached to a neutravidin scaffold.

19. The method of claim 17, wherein each barcoded pMHC construct comprises 5 identical pMHC monomers attached to a dextran scaffold.

20. The method of claim 1, wherein the sample comprising T lymphocytes is peripheral blood, cord blood, tissue biopsies, or liquid biopsies.

21. The method of claim 9, wherein the 5′ constant region comprises the nucleic acid sequence as set forth in SEQ ID NO: 1 (ACCTTAAGAGCCCACGGTTCC).

22. The method of claim 9, wherein the 3′ constant region comprises the nucleic acid sequence as set forth in SEQ ID NO: 2 (AAAGAATATACCC).

23-24. (canceled)

25. A DNA barcoded pMHC construct comprising at least one pMHC peptide covalently or non-covalently attached to a scaffold molecule, and at least one barcode covalently or non-covalently attached to the scaffold.

26-27. (canceled)

28. A method of manufacturing the DNA barcoded pMHC construct of claim 25, comprising:
(a) attaching a 4 or 5 pMHC peptides to a scaffold, wherein the scaffold is dextran or neutravidin; and
(b) attaching at least one DNA barcode to the scaffold, wherein the DNA barcode comprises SEQ ID NO: 3.
